(19) United States
(12) Patent Application Publication
(10) Pub. No.: US 2013/0310883 A1
(43) Pub. Date: Nov. 21, 2013

(54) EXPANDABLE BONE FIXATION ELEMENT

(71) Applicants: Mark M. Levy, Raanana (IL); Shimon Spector, Matan (IL)

(72) Inventors: Mark M. Levy, Raanana (IL); Shimon Spector, Matan (IL)

(21) Appl. No.: 13/472,650

(22) Filed: May 16, 2012

(51) Int. Cl. A61B 17/86

(52) U.S. Cl. 606/313

(57) ABSTRACT

A threaded bone fastener including a threaded shank, a head extending from the shank, one or more expandable elements, each having a distal end or a proximal end pivotally attached to the shank, the expandable elements being expandable radially outwards from the shank and contractible radially inwards towards the shank, wherein expansion and contraction of the expandable elements does not change an overall length of the shank.

### FIELD OF THE INVENTION

The present invention relates generally to expandable bone fixation devices, such as a threaded bone fastener with expandable elements.

### BACKGROUND OF THE INVENTION

Enhanced fixation of devices in the spine, such as pedicle screws, intervertebral devices, posterior element devices and vertebral replacement devices, is desired in certain situations, such as poor bone quality, disuse atrophy or revision cases with loosening.

Besides the vertebrae, other bones can benefit from more efficient fixation devices, such as for fixation of fractures, fusion of joints or connection of devices to bones (like plates, prostheses, dental implants and others).

### SUMMARY OF THE INVENTION

The present invention seeks to provide novel expandable bone fixation devices, as is described more in detail further below.

In embodiments of the invention, the fixation device includes a threaded bone fastener with expandable elements that expand radially outwards from a threaded shank (in parallel or not to the shank). In some embodiments, the expandable elements are threaded; in others they are not threaded. In some embodiments, the shank is fully threaded; in others the shank is only partially threaded.

The expandable elements originate from the main body of the threaded fastener. Upon expansion, these elements produce an additional fixation force, improving the stability of the device in the bone or adjacent tissue. In other situations, the expansion of the elements close the gap between two fragments of a fracture, two surfaces of a joint or a space between a device and a bone to which the device is attached by approximation of the two surfaces.

Some non-limiting features of the invention include maintaining a contiguous body to ensure mechanical integrity of the device. The outer surface of the device may include threads, a rough texture, a coating or any other treatment to improve fixation to hard or soft tissue as required. The material of the device can be any bone compatible material, biologic or synthetic, permanent or temporary, absorbable or not, or any combination thereof. Coating of the device, or portions thereof, is possible with natural or artificial substances to enhance bone adherence or improve biological processes; examples of this include hydroxyapatite, bone factors, antibiotics, antiseptic materials, drugs including bisphosphonates, metal coatings like tantalum, plasma coating, rough surface finishing and others,

The expandable elements may be cut or otherwise formed from the main body material, or may be added thereto. Additional sections or appendixes may be provided, which may be manufactured from the same or different materials as the main body.

The expandable elements can be an integral part of the device, hinged to the devices, bonded (e.g., glued), welded or attached with any means to the main body of the device. The overall length of the device is maintained because only a lateral section expands. Thus the device has a constant length, with or without threads, with better structural strength. Upon expansion, the expandable elements can adopt any shape, position or configuration.

The expandable elements can be actuated from within the device with an internal (built-in) actuator. For example, the expandable elements may be connected to a common central rod (e.g., threaded) that can be advanced or retracted in the shank of the device. The axial movement of the central rod expands the expandable elements radially outwards or contracts them radially inwards. In one embodiment, the actuation of the central actuator creates a relative void, proportional to the displacement that was used for the expansion. Such a void never compromises the contiguity or mechanical structural strength of the device.

Alternatively, the expandable elements can be manipulated with an external instrument. The actuator can be removed or left in place after the operation. The expandable elements can be locked after expansion or may be left without locking.

The expandable elements may undergo plastic deformation, elastic deformation, or no deformation or a combination thereof. The expansion increases the perimeter of the device at certain sections of it, in different shapes, increasing device-bone contact. If threaded, the expandable elements serve as an extension of the bolt/screw. The expandable elements may act as an integral nut on the shank, adding to the fixation force of the device.

The threaded fastener can have connectors, attachments or links to connect with other devices, such as in the same bone (e.g., vertebra), adjacent or distant bones.

The threaded fastener can be cannulated to be introduced over a guide wire for minimally invasive uses.

In some embodiments, both ends of the expandable element are connected to the shank of the device (particularly if the expandable element is unfolded or deformed in the process of expansion). In other embodiments, only one end of the expandable element is connected to the shank of the device. For example, the distal end may be connected while the proximal end slides axially with respect to the shank upon radially outward expansion of the expandable elements. Conversely, the proximal end may be connected while the distal end slides axially with respect to the shank upon radially outward expansion of the expandable elements.

In another embodiment, the expandable elements are additional sections added to the main body. The attachment of the expandable elements to the main body can be permanent (maintained for the functional life of the device) or temporary for detaching at some point in time. The detachment can be embodied as a loose or movable link, a breakable link, resorption inside the body or any other means.

### BRIEF DESCRIPTION OF THE DRAWINGS

The present invention will be understood and appreciated more fully from the following detailed description, taken in conjunction with the drawings in which:

FIGS. 1A and 1B are simplified perspective and side-view illustrations of a threaded bone fastener, constructed and operative in accordance with a non-limiting embodiment of the present invention, in a contracted orientation, including a pair of threaded expandable elements, and fully threaded shank.

FIGS. 1C and 1D are simplified perspective and side-view illustrations of the threaded bone fastener, in an expanded orientation.

FIGS. 2A and 2B are simplified side-view illustrations (rotated 90° from the orientation of FIG. 1B) of a threaded bone fastener, with expandable elements in accordance with two different embodiments of the present invention, in a contracted orientation.

FIGS. 3A and 3B are simplified illustrations of a threaded bone fastener, constructed and operative in accordance with another non-limiting embodiment of the present invention, in respective contracted and expanded orientations, including a pair of non-threaded expandable elements and fully threaded shank.

FIG. 4 is a simplified illustration of a threaded bone fastener, constructed and operative in accordance with yet another non-limiting embodiment of the present invention, in a contracted orientation, including a pair of non-threaded expandable elements and partially threaded shank.

FIGS. 5A and 5B are simplified illustrations of threaded bone fasteners, constructed and operative in accordance with other non-limiting embodiments of the present invention.

### DETAILED DESCRIPTION OF EXEMPLARY EMBODIMENTS

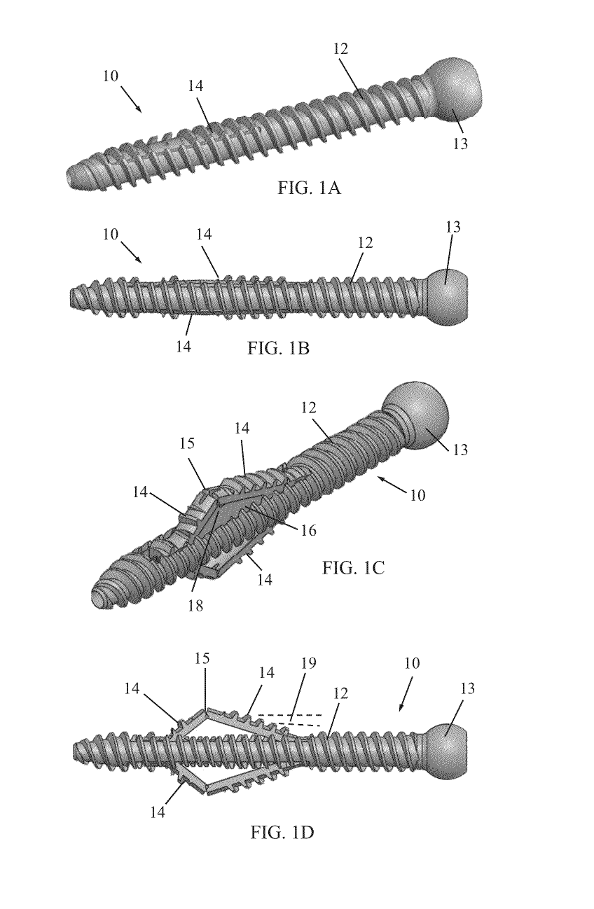

Reference is now made to FIGS. 1A-1D, which illustrate a threaded bone fastener 10, constructed and operative in accordance with a non-limiting embodiment of the present invention.

Threaded bone fastener 10 includes a shank 12, having a proximal portion 13 (such as a head of any shape or headless shank) and one or more (e.g., a pair of) expandable elements 14. Shank 12 may be threaded as shown. Alternatively, shank 12 may be non-threaded. In the illustrated embodiment, expandable elements 14 are threaded and are spaced 180° apart (but other spacings are also possible). Both proximal and distal ends of the expandable elements 14 are pivotally attached to shank 12. In the illustrated embodiment, the shank 12 is fully threaded.

The expandable elements 14 can be actuated with an internal (built-in) actuator 16 (FIG. 1C). For example, actuator 16 may be a central rod (e.g., threaded or non-threaded) with a cam 18 or otherwise attached to the expandable elements. Actuator 16 can be accessed through head 13 and turned (such as by a turning tool, e.g., screwdriver or socket wrench) or pushed/pulled with a linear actuator mechanism. In the case of a linear actuator, a shaped actuator (not necessarily of cylindrical shape) and a matching inner surface of the device may ensure there is no twisting and may increase torsional strength. Distal advancement of actuator 16 through shank 12 causes cam 18 to push against expandable elements 14 and expand them radially outwards (the state of FIGS. 1C-1D). Conversely, proximal retraction of actuator 16 releases the force of cam 18 against expandable elements 14 and causes them to contract radially inwards (the state of FIGS. 1A-1B). Other actuators may be used.

Alternatively, expandable elements 14 can be manipulated with an external instrument 19 (shown optionally in broken lines in FIGS. 1D). In other embodiments, actuator 16 can be detached and removed after expansion or be replaced by a reusable instrument. The actuator can be removed or left in place after the operation. Expandable elements 14 can be locked after expansion or may be left without locking.

As seen in FIGS. 1C-1D, expandable elements 14 may include a jointed structure, such as a pair of elements joined at a joint or hinge 15.

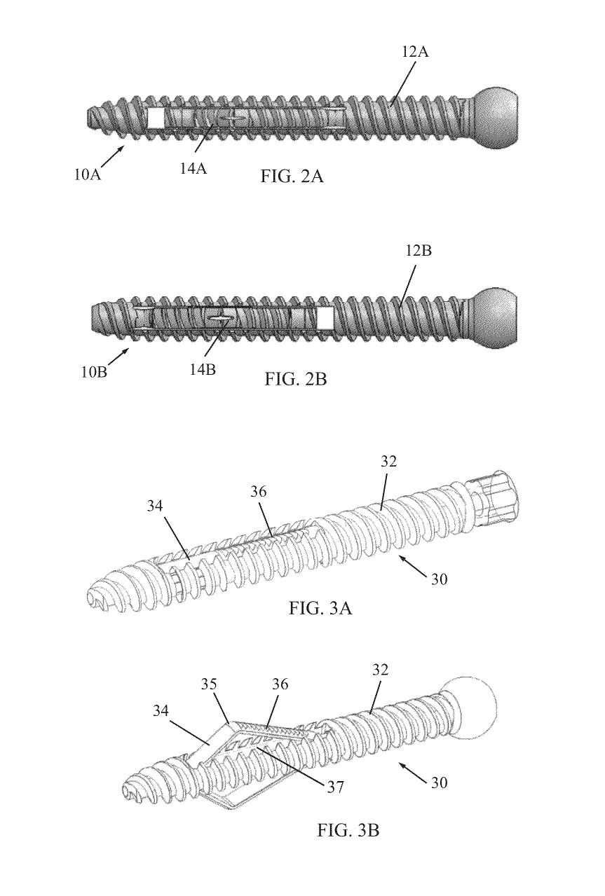

Reference is now made to FIGS. 2A and 2B, which illustrate threaded bone fasteners 10A and 10B, with expandable elements 14A and 14B, respectively.

In these embodiments, only one end of the expandable element is connected to the shank of the device. For example, in the embodiment of FIG. 2A, the proximal end of each expandable element 14A is connected to shank 12A, while the distal end slides axially with respect to shank 12A upon radially outward expansion of expandable elements 14A. Conversely, in the embodiment of FIG. 2B, the distal end of each expandable element 14B is connected to shank 12B, while the proximal end slides axially with respect to shank 12B upon radially outward expansion of expandable elements 14B.

In some or all of the above described embodiments, the actuation of the actuator may create a relative void, proportional to the displacement that was used for the expansion. Such a void never compromises the contiguity or mechanical structural strength of the device.

The expandable elements may undergo plastic deformation, elastic deformation, or no deformation or a combination thereof.

Reference is now made to FIGS. 3A and 3B, which illustrate a threaded bone fastener 30, constructed and operative in accordance with another non-limiting embodiment of the present invention. In this embodiment, there are non-threaded expandable elements 34 and a fully threaded shank 32. More specifically, as shown in the illustrated embodiment, one element 34 of each pair of expandable elements may be non-threaded, while another element 36 may be threaded; elements 34 and 36 meet at joint 35. Alternatively, both elements 34 and 36 can be non-threaded. In the embodiment of FIG. 3B, expandable elements 34 expand out of a blind groove 37 formed in shank 32.

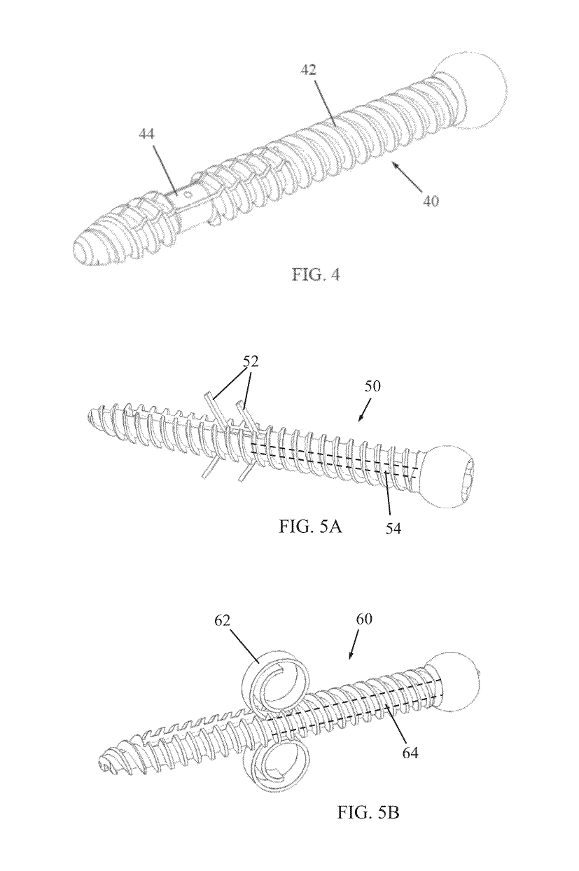

Reference is now made to FIG. 4, which illustrates a threaded bone fastener 40, constructed and operative in accordance with yet another non-limiting embodiment of the present invention, in a contracted orientation. In this embodiment, there are non-threaded expandable elements 44 and a partially threaded shank 42. A relatively small opening may be formed in shank 42 and the one or more expandable elements 44 may be formed from a same or different material than shank 42. The relatively small opening formed in the shank increases the mechanical strength of the shank. The opening may be through the thickness of the shank. Alternatively, instead of an opening, a blind groove or channel may be formed in the shank.

Reference is now made to FIGS. 5A and 5B, which illustrate threaded bone fasteners 50 and 60, respectively, constructed and operative in accordance with other non-limiting embodiments of the present invention. In threaded bone fastener 50, one or more expandable elements 52 are shaped as prongs that are attached to a central, elongate actuator 54 (e.g., bar or rod, the terms being used interchangeably for all the embodiments of the invention), which is a centrally located mechanism of fastener 50. Appropriate pushing or pulling of actuator 54 causes expandable elements 52 to expand outwards or contract inwards, respectively.

In threaded bone fastener 60, one or more expandable elements 62 are shaped as an elastic spiral, attached to a central, elongate actuator 64 (e.g., bar or rod), which is a centrally located mechanism of fastener 60. Without limitation, the spiral may be made of a shape memory alloy. Appropriate pushing or pulling of actuator 64 causes expandable elements 62 to expand outwards or contract inwards, respectively. In other embodiments, the expandable elements can expand outwards in a combination of radially-directed expansion plus another direction that is not radial with respect to the longitudinal axis of the shank, such as but not limited to, spiral, impeller-like shape, curved wing or any other fashion. This increases the bone contact in different manners other than merely radially about the shank.

Thus, these embodiments include expandable elements that have different shapes from previously described embodiments. In both of these embodiments, the expandable elements are connected to the central portion or mechanism of the fastener. The invention can be carried out with any shape and size of expandable element(s).

The scope of the present invention includes both combinations and subcombinations of the features described hereinabove as well as modifications and variations thereof which would occur to a person of skill in the art upon reading the foregoing description and which are not in the prior art.

What is claimed is:

1. A threaded bone fastener comprising:
a threaded shank comprising a proximal portion; and
one or more expandable elements, each having a distal end or a proximal end pivotally attached to said shank, said expandable elements being expandable radially outwards from said shank and contractible radially inwards towards said shank, wherein expansion and contraction of said expandable elements does not change an overall length of said shank.

2. The threaded bone fastener according to claim 1, wherein said expandable elements are threaded.

3. The threaded bone fastener according to claim 1, wherein said expandable elements are not threaded.

4. The threaded bone fastener according to claim 1, wherein said expandable elements are spaced 180° apart.

5. The threaded bone fastener according to claim 1, wherein said shank is fully threaded over its length.

6. The threaded bone fastener according to claim 1, wherein said shank is partially threaded over its length.

7. The threaded bone fastener according to claim 1, further comprising an actuator operative to expand said expandable elements radially outwards from said shank.

8. The threaded bone fastener according to claim 7, wherein said actuator comprises a central rod with a cam, wherein distal advancement of said actuator through said shank causes said cam to push against said expandable elements and expand them radially outwards.

9. The threaded bone fastener according to claim 7, wherein said actuator is accessible through said proximal portion.

10. The threaded bone fastener according to claim 1, wherein said expandable elements are lockable.

11. The threaded bone fastener according to claim 1, wherein a distal end of each said expandable element is connected to said shank, and a proximal end of each said expandable element slides axially with respect to said shank upon radially outward expansion of said expandable elements.

12. The threaded bone fastener according to claim 1, wherein a proximal end of each said expandable element is connected to said shank, and a distal end of each said expandable element slides axially with respect to said shank upon radially outward expansion of said expandable elements.

13. A bone fastener comprising:
a shank comprising a proximal portion;
one or more expandable elements, each having a distal end or a proximal end pivotally attached to said shank, said expandable elements being expandable radially outwards from said shank and contractible radially inwards towards said shank, wherein expansion and contraction of said expandable elements does not change an overall length of said shank;
an actuator operative to expand said expandable elements radially outwards from said shank, wherein said actuator comprises a central rod with a cam, wherein distal advancement of said actuator through said shank causes said cam to push against said expandable elements and expand them radially outwards, and wherein said actuator is accessible through said proximal portion.

14. A threaded bone fastener comprising:
a threaded shank comprising a proximal portion; and
one or more expandable elements attached to a central, elongate actuator, wherein pushing or pulling of said actuator causes said expandable elements to expand outwards or contract inwards, and wherein expansion and contraction of said expandable elements does not change an overall length of said shank, and wherein said actuator comprises a central portion of said fastener.

15. The threaded bone fastener according to claim 14, wherein said one or more expandable elements comprise prongs.

16. The threaded bone fastener according to claim 14, wherein said one or more expandable elements comprise an elastic spiral.

17. The threaded bone fastener according to claim 14 wherein said shank is at least partially threaded.

18. The threaded bone fastener according to claim 1, wherein said one or more expandable elements expand outwards in a combination of radially-directed expansion plus another direction that is not radial with respect to a longitudinal axis of said shank.
